SEQUENCE LISTING (4 sequences)

SEQ ID NO: 1 - DNA, 23 bp, Artificial
  FORWARD PRIMER
gctaatacatgctgaaaacccca

SEQ ID NO: 2 - DNA, 23 bp, Artificial
  REVERSE PRIMER
gcgggtcataatagaaacaccgc

SEQ ID NO: 3 - DNA, 22 bp, Artificial
  FORWARD PRIMER
taaatagcccggtccgcattcg

SEQ ID NO: 4 - DNA, 19 bp, Artificial
  REVERSE PRIMER
tcccctgagccagtccgaa

(19) United States
(12) Patent Application Publication
(10) Pub. No.: US 2010/0012495 A1
(43) Pub. Date: Jan. 21, 2010

(54) Set of Novel Oligonucleotide Primers and the Method for the Detection of Aspergillus Ochraceux Thereby

(76) Inventors: Anand Sreenivasan, Karnataka (IN); Ratirao Eddiya, Karnataka (IN)

Correspondence Address: ABELMAN, FRAYNE & SCHWAB, 666 THIRD AVENUE, 10TH FLOOR, NEW YORK, NY, 10017, US

(21) Appl. No.: 12/226,022

(22) PCT Filed: Sep. 28, 2007
(86) PCT No.: PCT/IN2007/000449

§ 371 Date: Sep. 11, 2009

(30) Foreign Application Priority Data
Dec. 7, 2006 (IN) 2624/DEL/2006

(51) Int. Cl. G01N 27/26; C07H 21/04; C12Q 1/68

(52) U.S. Cl. 204/461; 536/24.33; 435/6

(57) ABSTRACT

The present invention relates to a process for the detection of Aspergillus ochraceus by biotechnological approach. This invention particularly relates to the development of a process for the detection of species belonging to Aspergillus ochraceus group by PCR/ multiplex PCR technique. More particularly the present invention relates to a set of novel oligonucleotide primers and the method for the detection of Aspergillus ochraceus thereby.

The present invention relates to a process for the detection of Aspergillus ochraceus by biotechnological approach. This invention particularly relates to the development of a process for the detection of species belonging to Aspergillus ochraceus group by PCR/ multiplex PCR technique. More particularly the present invention relates to a set of novel oligonucleotide primers and the method for the detection of Aspergillus ochraceus thereby.

### BACKGROUND OF INVENTION

Aspergillus ochraceus group of fungi belonging to Genus Aspergillus section Circumdati are known to produce the mycotoxin ochratoxin A. Ochratoxins belong to pentaketide group of mycotoxins, which consists of isocoumarin molecule linked to phenylalanine through, amide linkage (Vander Merwe et al, 1965). Ochratoxins are known nephrotoxic agent, apart from that they have teratogenic, immunosuppressive and carcinogenic properties (Krogh, et al 1992). It has also been classified as possible human carcinogen (group 2B) by international agency of cancer (Kuiper Godman, et al., 1996).

Ochratoxin A was initially known to be produced by Aspergillus ochraceus Wilhelm and Penicillium verrucossum (Pitt, 1987: Ciegler, 1992). But later studies have shown that other species of Aspergillus ochraceus group—Aspergillus melleus, A. sulphureus, A. sclerotiorum, A. auricomus, A. alliaceus and A. ostianus (Varga et al., 1996) as ochratoxin A producer. Aspergillus ochraceus group of fungi is widely distributed in dried foods like beans, dried fruits, nuts including peanuts, betal nuts, wide range of cereals including barley, wheat flour and rice (Pitt and Hocking, 1985: Varga et al, 1996). Aspergillus ochraceus has been considered principle organism responsible for ochratoxin A contamination in tropical region and Penicillium verrucossum as the principle organism responsible for ochratoxin A contamination in temperate regions (Pitt, 1987).

Mycotoxigenic fungi are widely distributed in various food commodities and mycotoxins contribute to loss of about 25% food grains produced globally. The level of fungal infestation and identification of the governing species are important. parameters, which would give an indication of the quality of the material and future potential for the presence of ochratoxins. Mold counts are a part of quality control assurance for foods. This method is time consuming, labour intensive, costly, requires facilities, and mycological expertise. In this direction, DNA-based detection method like PCR is more sensitive, specific and has been employed for the detection of various pathogenic and mycotoxigenic fungi.

Sequence variations in the rDNA gene have been used for phylogenetic studies and various primers have been designed for specific detection of various organisms. The main reasons for the popularity of rDNA are that it is a multiple-copy, non-protein coding gene and are present in all organisms with a common evolutionary origin. Fungal rRNA genes are organized in units each of which encodes three mature subunits of 18S, 5.8S, and 28S respectively. Two internal transcribed spacers, ITS1 and ITS2 separate these subunits (White et al., 1990). The ITS regions are generally used for differentiation at the species level Josep et al (1999). The 18S rDNA is known evolve relatively slow compared to internal transcribed spacers and therefore suitable for development of genus or group level probes.

References may be made to the work of Wu, et al (2003) wherein 33 oligonucleotide probes designed based on the sequence variation in 18S rRNA gene has been reported as genus or species specific. The probes Asp-3 and Asp-4 have been reported as specific to Aspergillus flavus, A. fumigatus, A. niger, A. ochraceus, and A. versicolor but could not differentiate among these species.

References may be made to the work of Johanson et al (1998), wherein they have reported PCR method for detection of Rhizoctonia solani, R. oryzae and R. oryzae saliva using specific primers designed based on the sequence variation in ITS1 and ITS2 region among these species.

References may be made to the work of Zur et al (2003) wherein they have reported oligonucleotide primers specific for Alternaria alternata and Alternata solani based on sequence variation in the sequence of ITS1 and ITS2 region.

References may be made to the work of Accensi et al (1999), wherein a PCR method has been described to differentiate A. niger from A. tubingensis based on variation of single nucleotide in ITS region of these species.

References may be made to the work of Colombo et al (2003) wherein they have described method for detection of Penicillium auratiogriseum based on sequence variation in ITS1 and ITS2 region which can be differentiated from other fungal species by PCR and restriction fragment length polymorphism (RFLP) technique.

References may be made to the work of Sandhu et al (1995) wherein they have reported 21 oligonucleotide probes specific for A. flavus, A. fumigatus, A. niger, A. glaucus and A. terreus spp of Aspergillus. Their list do not include A. ochraceus group.

References may be made to the work of Martin et al (2000) wherein they have reported oligonucleotide probes for detection of fungal pathogens by PCR based line probe assay (LIPA). They have reported method for detection of A. fumigatus, A. flavus, A. versicolor and A. nidulans.

The drawbacks of these references are that no attempts have been made for specific detection of Aspergillus ochraceus species and the traditional methods are less sensitive, time-consuming and lack consistency. The present invention enables the detection of Aspergillus ochraceus species by PCR, using primer specific for 18S rRNA gene. This method has application in detection of ochratoxin producing Aspergillus ochraceus in food system.

In U.S. Pat. No. 6,180,339 Nucleic acid probes and primers are described for detecting fungi that cause disease in humans and animals, as well as spoilage of food and beverages. These probes can detect rRNA, rDNA or polymerase chain reaction products from a majority of fungi in clinical, environmental or food samples. Nucleic acid hybridization assay probes specific for Acremonium sp., Aspergillus clavatus, Aspergillus flavus, Aspergillus fumigatus, Aspergillus glaucus, Aspergillus nidulans, Aspergillus niger, Aspergillus ochraceus, Aspergillus terreus, Aspergillus unguis, Aspergillus ustus, Beauveria sp., Bipolaris sp., Blastoschizomyces sp., Blastomyces dermatitidis, Candida albicans, Candida glabrata, Candida guilliermondii, Candida kefyr, Candida krusei, Candida lusitaniae, Candida parapsilosis, Candida tropicalis, Chrysosporium sp., Cladosporium sp., Coccidioides immitis, Cryptococcus neoformans var gattii serotype B, Cryptococcus neoformans serotype A, Cryptococcus laurentii, Cryptococcus terreus, Curvularia sp., Fusarium sp., Filobasidium capsuligenum, Filobasidiella (Cryptococcus) neoformans var bacillispora serotype C, Filobasidiella (Cryptococcus) neoformans var neoformans serotype D, Filobasidium uniguttulatum, Geotrichum sp., Histoplasma capsulatum, Malbranchea sp., Mucor sp., Paecilomyces sp., Paracoccidioides brasiliensis, Penicillium species, Pneumocystis carinii, Pseudallescheria boydii, Rhizopus sp., Sporothrix schenkii, Scopulariopsis brevicaulis, Scopulariopsis brumpti, Saccharomyces cerevisiae, and Trichosporon beigelii are also described. The present invention enables the detection of Aspergillus ochraceus species by PCR, using primer specific for 18S rRNA gene. This method has application in detection of ochratoxin producing Aspergillus ochraceus in food system.

In U.S. Pat. No. 5,763,169 Nucleic acid probes and primers are described for detecting fungi that cause disease in humans and animals, as well as spoilage of food and beverages. These probes can detect rRNA, rDNA or polymerase chain reaction products from a majority of fungi in clinical, environmental or food samples. Nucleic acid hybridization assay probes specific for Aspergillus fumigatus, Blastomyces dermatitidis, Candida albicans, Coccidioides immitis, Cryptococcus neoformans, Histoplasma capsulatum, Aspergillus flavus, Aspergillus glaucus, Aspergillus niger, Aspergillus terreus, Candida glabrata, Candida guilliermondii, Candida kefyr, Candida krusei, Candida lusitaniae, Candida parapsilosis, Candida tropicalis, Pseudallescheria boydii, and Sporothrix schenckii are also described. The present invention enables the detection of Aspergillus ochraceus species by PCR, using primer specific for 18S rRNA gene. This method has application in detection of ochratoxin producing Aspergillus ochraceus in food system.

In U.S. Pat. No. 5,800,997 internal Transcribed Spacer (ITS) DNA sequences from the ribosomal RNA gene region are described for different species and strains of Helminthosporium carbonum, Helminthosporium turcicum, Helminthosporium maydis, Cercospora zeae-maydis, Kabatiella zeae and Puccinia sorghi. Specific primers from within these sequences are identified as being useful for the identification of the fungal isolates using PCR-based techniques. The present invention enables the detection of Aspergillus ochraceus species by PCR, using primer specific for 18S rRNA gene. This method has application in detection of ochratoxin producing Aspergillus ochraceus in food system.

In U.S. Pat. No. 5,792,611 root rots are serious conifer nursery diseases. At present, seedlings are inspected visually to determine whether they show symptoms of disease. Cylindrocarpon destructans and Cylindrocarpon floridanum are two important causal agents of root rots. A substantially more efficient and reliable method of detecting the presence of these fungi directly from infected tissue involves the use of a set of DNA primers which can be used in conjunction with the polymerase chain reaction (PCR) to create species specific probes for detecting species of each fungi. The present invention enables the detection of Aspergillus ochraceus species by PCR, using primer specific for 18S rRNA gene. This method has application in detection of ochratoxin producing Aspergillus ochraceus in food system.

In U.S. Pat. No. 6,080,543 Unique DNA sequences are provided which are useful in identifying different pathogenic fungi, such as those which infect grape plants. These unique DNA sequences can be used to provide oligonucleotide primers in PCR based analysis for the identification of fungal pathogens. The DNA sequences of the present invention include the internal transcribed spacer (ITS) of the ribosomal RNA gene regions of particular fungal pathogens, as well as oligonucleotide primers which are derived from these regions which are capable of identifying the particular pathogen. The present invention enables the detection of Aspergillus ochraceus species by PCR, using primer specific for 18S rRNA gene. This method has application in detection of ochratoxin producing Aspergillus ochraceus in food system.

In U.S. Pat. No. 5,580,971 the invention includes methods for the detection of a particular genus or species of fungus in a biological sample. Some of these methods make use of a solid support-polynucleotide structure. This structure includes a solid support having immobilized thereto a polynucleotide probe that is complementary to a sequence of ribosomal RNA (rRNA) specific to the particular species of fungus. An rRNA sequence from the particular species of fungus is hybridized to the first probe, and a second polynucleotide probe is also hybridized to the rRNA. The present invention enables the detection of Aspergillus ochraceus species by PCR, using primer specific for 18S rRNA gene. This method has application in detection of ochratoxin producing Aspergillus ochraceus in food system.

In U.S. Pat. No. 5,324,632 Nucleic acid probes are described for detecting fungi capable of causing fungal septicemia or capable of causing food spoilage. The preferred probes are complementary to ribonucleic acid sequences found in numerous fungi and absent in animal or plant genomes. As such, these probes can detect the rRNA, rDNA, or polymerase chain reaction amplification products from the majority of fungal species. The detection of etiological agents of human fungemia, the clinical diagnosis of this disease and the direct evaluation of food or beverage fungal content utilizing rRNA or rDNA probes is now possible. The present invention enables the detection of Aspergillus ochraceus species by PCR, using primer specific for 18S rRNA gene. This method has application in detection of ochratoxin producing Aspergillus ochraceus in food system.

### SUMMARY OF THE PRESENT INVENTION

The main object of the present invention is to provide a PCR/ multiplex PCR method for the detection of Aspergillus ochraceus group of fungi comprising the following species Aspergillus ochraceus, Aspergillus melleus, A. sulphureus, A. sclerotiorum, A. auricomus, and A. ostianus, which obviates the drawbacks detailed above. The present invention comprises primer pairs designed for amplification of 18S rRNA gene of the target organism Aspergillus ochraceus. The method also uses PCR/multiplex PCR conditions specific for the detection of 18S rRNA gene in the Aspergillus ochraceus in the pure culture and food system, making the method a very sensitive one.

### BRIEF DESCRIPTION OF THE DRAWINGS

The figures represent the PCR amplification products of genomic DNA of Aspergillus ochraceus group of fungi, which produce ochratoxin A.

FIG. 1 represents PCR Amplification of Aspergillus ochraceus with OT1 primers. Lane 1-Negative control, 2-Aspergillus ochraceus MTCC 1877, 3-Aspergillus melleus CFR 226, 4-Aspergillus sulphureus CFR 230, 5-Aspergillus sclerotiorum CFR 227, 6-Aspergillus auricomus CFR 229, 7-Aspergillus ostianus CFR 228, M-100 bp ladder;

FIG. 2, represents PCR amplification of A. ochraceus group fungi with OT2 primers. Lane 1-Negative control, 2-Aspergillus ochraceus CFR 221, 3-Aspergillus ochraceus MTCC 1877, 4-Aspergillus melleus CFR 226, 5-Aspergillus sulphureus CFR 230, 6-Aspergillus sclerotiorum CFR 227, 7-Aspergillus auricomus CFR 229, 8- Aspergillus ostianus CFR 228, M-100 bp ladder;

FIG. 3 represents PCR amplification of A. ochraceus group fungi with OT2 primers. Lane 1-Negative control, 2-Aspergillus ochraceus CFR 221, 3-Aspergillus ochraceus MTCC 1877, 4-Aspergillus melleus CFR 226, 5-Aspergillus sulphureus CFR 230, 6-Aspergillus sclerotiorum CFR 227, 7-Aspergillus auricomus CFR 229, 8-Aspergillus ostianus CFR 228, M-100 bp ladder; and

FIG. 4, represents Amplification of Aspergillus ochraceus with OT1 and OT2 primers Lane 1-Negative control, 2-Maize (10⁶spores/gm) amplified with OT1, 3-Maize (10⁶spores/gm) amplified with OT1, 4-Maize (10⁶spores/gm) amplified with OT1 and OT2, M-Molecular size marker.

### DESCRIPTION OF THE INVENTION

An embodiment of the present invention provides a set of novel oligonucleotides primers selected from the group consisting of OT1F, OT1R, OT2F and OT2R having the following respective sequences:

SEQ ID NO: 01

OT1F-5′ GCTAATACATGCTGAAAACCCCA 3′

SEQ ID NO: 02

OT1R-5′ GCGGGTCATAATAGAAACACCGC 3′

SEQ ID NO: 03

OT2F-5′ TAAATAGCCCGGTCCGCATTCG 3′

SEQ ID NO: 04

OT2R-5′ TCCCCTGAGCCAGTCCGAA 3′

useful for amplification of target nucleotide fragment forming a part of 18S rRNA
gene in species belonging to Aspergillus ochraceus (MTCC No: 1877) group.

The primer pair OT1F (SEQ ID NO: 01) and OT1R (SEQ ID NO: 02) is useful for amplification of 906 bp nucleotide fragment forming a part of 18S rRNA gene in species belonging to Aspergillus ochraceus (MTCC No: 1877) group. The primer pair OT2F (SEQ ID NO: 03) and OT2R (SEQ ID NO: 02) is useful for amplification of 353 bp nucleotide fragment forming a part of 18S rRNA gene in species belonging to Aspergillus ochraceus (MTCC No: 1877) group. The said amplification is achieved by Polymerase Chain Reaction (PCR), Rolling Circle Amplification (RCA), Strand Displacement Amplification (SDA), Nucleic Acid Sequence Based Amplification (NASBA).

The said amplification is useful for various applications selected from the group consisting of detecting, quantifying, screening, quality-monitoring and combination thereof.

The said primer sequences and/or parts thereof are useful as probes for the detecting nucleotide fragment forming a part of 18S rRNA gene in species belonging to Aspergillus ochraceus (MTCC No: 1877) Group.

Another embodiment of the present invention provides a method for the detection of Aspergillus ochraceus (MTCC No: 1877) group, using oligonucleotide primers, which comprises the steps of:
(a) isolation, by a known method, of DNA from the source sample contaminated with Aspergillus ochraceus (MTCC No: 1877) group;
(b) subjecting the DNA isolated in step(a) to Polymerase Chain Reaction (PCR) amplification using at least one pair of the primers selected from the group consisting of pairs OT2F (SEQ ID NO: 03) and OT2R (SEQ ID NO: 02) OT1F (SEQ ID NO: 01), OT1R (SEQ ID NO: 02) and combination thereof having the following nucleotide sequence:

SEQ ID NO: 01

OT1F-5′ GCTAATACATGCTGAAAACCCCA 3′

SEQ ID NO: 02

OT1R-5′ GCGGGTCATAATAGAAACACCGC 3′

SEQ ID NO: 03

OT2F-5′ TAAATAGCCCGGTCCGCATTCG 3′

SEQ ID NO: 04

OT2R-5′ TCCCCTGAGCCAGTCCGAA 3′

useful for amplification of target nucleotide fragment forming a part of 18S rRNA gene in species belonging to Aspergillus ochraceus (MTCC No: 1877) group;AND
(c) detecting the Aspergillus ochraceus (MTCC No: 1877) group by visualizing the product(s) of said Polymerase Chain Reaction (PCR) amplification product(s) corresponding to target nucleotide fragment forming a part of 18S rRNA gene in species belonging to Aspergillus ochraceus (MTCC No: 1877) group.

The said Polymerase Chain Reaction (PCR) amplification used has the following parameters: PCR reaction mixture:- total reaction volume of 25 μl containing 1× PCR buffer (10 mM Tris-HCl, pH 9.0, 50 mM MgCl2, 0.01% gelatin); Deoxyribo nucleoside triphosphate: 12.5 μM each; Primers: 10 pmol each; Template DNA: 25 ng; Taq DNA polymerase: 1 unit; and Sterile ultrafiltered water: added to make up the total reaction volume of 25 μl; AND

Thermal parameters:—initial denaturation at 90°-98° C. for 2-8 min; initial amplification of 6-10 cycles each with a denaturation at 93°-95° C. for 20-40 sec, annealing at 68° to 72° for 35 to 55 seconds and an extension at 70° to 74° C. for 60 to 90 seconds followed by amplification of 20-24 cycles each with a denaturation at 93°-95° C. for 20-40 sec, annealing at 58° to 62° for 35 to 55 seconds and an extension at 70° to 74° C. for 60 to 90 seconds and a final extension at 68° to 76° C. for 6 to 10 minutes.

The said detection is carried out by analysing the PCR products in 1.0-1.2% agarose gel and subjected to electrophoresis for 1.5-2.5 h at about 100 volts in about 1× TAE buffer in order to get bands; staining the bands with ethidium bromide (0.5 μg/ml) followed by de-staining with distilled water and examination on a UV-transilluminator for appearance of bands , against 100 bp ladder used as molecular weight marker, corresponding to target nucleotide fragment forming a part of 18S rRNA gene in species belonging to Aspergillus ochraceus (MTCC No: 1877) group.

Yet another embodiment of the present invention provides that an amplification of 18S rRNA gene of Aspergillus ochraceus group is effected by about 30 amplification cycles comprising of about 8 cycles each having denaturation for about 30 sec at about 94° C., primer annealing for about 45 seconds at about 70° C., extension for about 75 seconds at about 72° C. followed by about 22 cycles each having denaturation for about 30 sec at about 94° C., primer annealing for about 45 seconds at about 60° C., extension for about 75 seconds at about 72° C. and final extension for about 8 minutes at about 72° C. In an embodiment of the present invention PCR used is performed by multiplex PCR in order to obtain the following amplification products:

| Primers used | Amplification product size |
|---|---|
| OT1F and OT1R | 906 bp |
| OT2F and OT2R | 353 bp |
| OT1F and OT2R | 1532 bp |

In an embodiment of the present invention the source sample used is selected from the group consisting of any food systems, foods, beverages, grains, fruits, vegetables, oil seeds, oil and mixture thereof.

In another embodiment of the present invention the source sample used is selected from the group consisting of any baked- or un-baked foods, food-systems, vegetables grains, fruits, vegetables, oil seeds, oil and mixture thereof.

In yet another embodiment of the present invention the source sample used is selected from the group consisting of soil, water, air and mixture thereof.

Yet another embodiment of the present invention provides a kit for amplifying or quantifying a nucleic acid target comprising at least one pair of the primers selected from the group consisting of pairs OT2F (SEQ ID NO: 03) and OT2R (SEQ ID NO: 02) OT1F (SEQ ID NO: 01), OT1R (SEQ ID NO: 02) and combination thereof having the following nucleotide sequence:

SEQ ID NO: 01

OT1F-5′ GCTAATACATGCTGAAAACCCCA 3′

SEQ ID NO: 02

OT1R-5′ GCGGGTCATAATAGAAACACCGC 3′

SEQ ID NO: 03

OT2F-5′ TAAATAGCCCGGTCCGCATTCG 3′

SEQ ID NO: 04

OT2R-5′ TCCCCTGAGCCAGTCCGAA 3′

useful for amplification of target nucleotide fragment forming a part of 18S rRNA gene in species belonging to Aspergillus ochraceus (MTCC No: 1877) group.

Accordingly the present invention provides a method for the detection of Aspergillus ochraceus group in food systems, which comprises;
a. Oligonucleotide primers for specific amplification of 18S rRNA gene in Aspergillus ochraceus designed based on the sequence comparison of 18S rRNA gene sequences listed in table.1 using gene multiple sequence alignment computer software programmes DIALIGN 2.2.1 and Biological sequence alignment editor and analysis program for Windows 95/98/NT (BioEdit version 5.0.9), sequence of which is given below:

1. OT1F-5′ GCTAATACATGCTGAAAACCCCA 3′
2. OT1R-5′ GCGGGTCATAATAGAAACACCGC 3′
3. OT2F-5′ TAAATAGCCCGGTCCGCATTCG 3′
4. OT2R-5′ TCCCCTGAGCCAGTCCGAA 3′

b. A method for detection of Aspergillus ochraceus group of fungi using primers specific for 18S rRNA gene wherein the primer set OT1 and OT2 amplifies 906 bp and 353 bp fragment of 18S rRNA gene respectively.
c. Extraction of template DNA by from moulds isolated from varied food sources.
d. Extraction of template DNA by standard method from moulds may be effected by treating the mycelia mass in Tris-EDTA (50 mM; 5 mM) buffer containing 2% Sodium dodecyl Sulphate (SDS) with three cycles of freezing at −20° C. for 10 minutes and thawing for 5 minutes. The treated mycelium may be taken in 1.5 ml microcentrifuge tube and incubated at 65° C. for 60 min followed by extraction using phenol: chloroform: isoamyl alcohol at 25:24:01.
e. The PCR reaction mixture in a total volume of 25 μl may consist of buffer 2.5 μl (10 mM Tris-HCl, pH9.0, 50 mM KCl, 1.5 mM MgCl2, 0.01% gelatin), dNTP mix (12.5 uM each) 0.51 μl, Taq-DNA Polymerase 1 Unit, specific primer—forward 1 μl, reverse 1 μl, 25 ng template DNA 1 μl and sterile water to make upto 25 ul.
f. Detection of Aspergillus ochraceus group of fungi by amplification of target gene may be effected from an initial denaturation at 93-95° C. for 2 to 8 min, initial amplification of 6-10, cycles each with a denaturation at 93°-95° C. for 20-40 sec, annealing at 68° to 72° for 35 to 55 seconds and an extension at 70° to 74° C. for 60 to 90 seconds followed by amplification of 20-24 cycles each with a denaturation at 93°-95° C. for 20-40 sec, annealing at 58° to 62° for 35 to 55 seconds and an extension at 70° to 74° C. for 60 to 90 seconds and a final extension at 68° to 76° C. for 6 to 10 minutes.
g. The analysis of the PCR product may be achieved by following the standard procedure using 1.2-1.8% agarose gel electrophoresis and visualization of PCR product by staining with 0.5 μg/ml ethidium bromide and observation in a UV Transilluminator.

In an embodiment of the present invention, amplification was performed in total reaction volume of 25 μl, which contained 2.5 ul of PCR buffer (10 mM Tris-HCl, pH9.0, 50 mM KCl, 1.5 mM MgCl2, 0.01% gelatin), 12.5 μM each of deoxynucleotide phosphate, 10 pmol of each primer, and one unit of Taq DNA polymerase 25 ng template DNA and sterile ultra filtered water.

Template DNA was initially denatured at 95° C. for 5 minutes. Subsequently a total of 30 amplification cycles was carried out in a programmable Thermocycler with two sets of amplification cycles. The initial 6-10 cycles were programmed as follows: denaturation for 30 sec at 94° C., primer annealing for 45 seconds at 70° C., extension for 75 seconds at 72° C. followed by 20-24 cycles each with denaturation for 30 sec at 94° C., primer annealing for 45 seconds at 60° C., extension for 75 seconds at 72° C. and final extension for 8 minutes at 72° C.

The patent relates to. PCR/ multiplex PCR method for detection of ochratoxin A producing species of Aspergillus ochraceus fungi such as Aspergillus ochraceus, A. melleus, A. sulphureus, A. sclerotiorum, A. auricomus, A. alliaceus and A. ostianus. Polymerase chain reaction was used to selectively amplify 18S rRNA gene of Aspergillus ochraceus and related species. Aspergillus ochraceus species grown on a semi-synthetic medium was used for the isolation of template DNA. The PCR reaction mixture and amplification conditions were optimized for specific amplification. Visualization of PCR products revealed that by the method followed, it is possible to specifically detect Aspergillus ochraceus species isolated from different sources.

The novelty of this method is the use of the designed primers for the detection of Aspergillus ochraceus species by PCR and multiplex PCR. The PCR method is rapid and sensitive making it possible to detect Aspergillus ochraceus group of fungi isolated from varied food sources.

The following examples are given by way of illustrations of the present invention and therefore should not be construed to limit the scope of the present invention.

### Example 1

An oligonucleotide primer for specific amplification of 18S rRNA gene was designed based on the sequence comparison of 18S rRNA gene sequences listed in table.1 using gene multiple sequence alignment computer software programmes DIALIGN 2.2.1 and Biological sequence alignment editor and analysis program for Windows 95/98/NT (BioEdit version 5.0.9). This primer set amplifies 906 base pair (bp) fragment of the gene, the sequence of which is given below.

1. OT1F-5′ GCTAATACATGCTGAAAACCCCA 3′
2. OT1R-5′ GCGGGTCATAATAGAAACACCGC 3′

Aspergillus ochraceus species were grown on semi-synthetic medium for 34 days. Sterilization of media and other solutions was by autoclaving for 20 minutes at 121° C. The 50ml potato dextrose broth (PDB) medium was inoculated with 1 ml mycelia suspension of different isolates of Aspergillus ochraceus group and other fungal species as mentioned in table.2. The flasks were incubated at ambient temperature of 26° to 28° C. under stationary conditions for 48-72 h. The mycelia from the culture broth were separated by centrifugation.

The DNA from the mycelium was extracted for PCR by the method of Lee et al (1998). Amplification was performed in total reaction volume of 25 μl, which contained 1× PCR buffer (10 mM Tris-HCl, pH9.0, 50 mM KCl, 1.5 mM MgCl2, 0.01% gelatin), 12.5 μM each of deoxynucleotide phosphate, 10 pmol of each primer, and one unit of Taq DNA Polymerase, 10 ng template DNA and sterile ultra filtered water. Template DNA was initially denatured at 95° C. for 5 minutes. Subsequently a total of 30 amplification cycles was carried out in a programmable thermocycler with an initial 8 cycles each with a denaturation for 30 sec at 94° C., primer annealing for 45 seconds at 70° C., extension for 75 seconds at 72° C. followed by 22 cycles each with a denaturation for 30 sec at 94° C., primer annealing for 45 seconds at 60° C., extension for 75 seconds at 72° C. and final extension for 8 minutes at 72° C. PCR products were analyzed by standard procedure using agarose gel electrophoresis. Gel was stained with ethidium bromide (0.5 μg/ml), destained with distilled water and examined on a UV-transilluminator. A 100 bp ladder was used as a molecular size marker. The amplification profile of the gel was documented in a CCD-Camera based gel documentation system. The data presented in FIG. 1 indicates specific amplification of the target 18S rDNA gene in Aspergillus ochraceus species by OT1 primer set However, none of the other fungal species (data not shown) gave positive amplification for OT1 primer.

### Example 2

An oligonucleotide primer for specific amplification of 18S rRNA gene was designed based on the sequence comparison of 18S rRNA gene sequences listed in table.1 using gene multiple sequence alignment computer software programmes DIALIGN 2.2.1 and Biological sequence alignment editor and analysis program for Windows 95/98/NT (BioEdit version 5.0.9). This primer set amplifies 353 base pair (bp) fragment of the 18S rDNA, the sequence of which is given below:

1. OT2F-5′ TAAATAGCCCGGTCCGCATTCG 3′
2. OT2R-5′ TCCCCTGAGCCAGTCCGAA 3′

Aspergillus ochraceus species were grown on semi-synthetic medium for 3-4 days. Sterilization of media and other solutions was by autoclaving for 20 minutes at 121° C. The 50 ml potato dextrose broth (PDB) medium was inoculated with lml mycelia suspension of different isolates of Aspergillus ochraceus group and other fungal species as mentioned in table.2. The flasks were incubated at ambient temperature of 26° to 28° C. under stationary conditions for 48-72 h. The mycelia from the culture broth were separated by centrifugation.

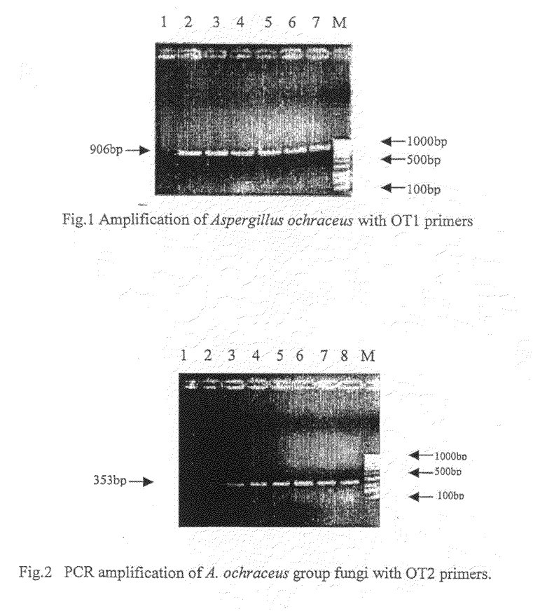

The DNA from the mycelium was extracted for PCR by the method of Lee et al (1998). Amplification was performed in total reaction volume of 25 μl, which contained 1× PCR buffer (10 mM Tris-HCl, pH9.0, 50 mM KCl, 1.5 mM MgCl2, 0.01% gelatin), 12.5 μM each of deoxynucleotide phosphate, 10 pmol of each primer, and one unit of Taq DNA Polymerase, 10 ng template DNA and sterile ultra filtered water. Template DNA was initially denatured at 95° C. for 5 minutes. Subsequently a total of 30 amplification cycles was carried out in a programmable thermocycler with an initial 8 cycles each with a denaturation for 30 sec at 94° C., primer annealing for 45 seconds at 70° C., extension for 75 seconds at 72° C. followed by 22 cycles each with a denaturation for 30 sec at 94° C., primer annealing for 45 seconds at 60° C., extension for 75 seconds at 72° C. and final extension for 8 minutes at 72° C. PCR products were analyzed by standard procedure using agarose gel electrophoresis. Gel was stained with ethidium bromide (0.5 μg/ml), destained with distilled water and examined on a UV-transilluminator. A 100 bp ladder was used as a molecular size marker. The amplification profile of the gel was documented in a CCD-Camera based gel documentation system. The data presented in FIG. 2. indicates specific amplification of the target 18S rRNA gene in Aspergillus ochraceus species by OT2 primer set However, none of the other fungal species (data not shown) gave positive amplification for OT2 primer.

### Example 3

The application of the primers mentioned in Example.1 and Example.2 for multiplex PCR is illustrated in this section. The primer set OT1 and OT2 amplifies 906 bp and 353 bp of 18S rRNA respectively, the sequences of which are given below. When the primer sets OT1 and OT2 are used together in multiplex PCR a product of 1532 bp is obtained as a result of amplification of DNA affected by the interaction of OT1F and OT2R primer set.

SEQ ID NO: 01

OT1F-5′ GCTAATACATGCTGAAAACCCCA 3′

SEQ ID NO: 02

OT1R-5′ GCGGGTCATAATAGAAACACCGC 3′

SEQ ID NO: 03

OT2F-5′ TAAATAGCCCGGTCCGCATTCG 3′

SEQ ID NO: 04

OT2R-5′ TCCCCTGAGCCAGTCCGAA 3′

Aspergillus ochraceus species were grown on semi-synthetic medium for 3-4 days. Sterilization of media and other solutions was by autoclaving for 20 minutes at 121° C. The 50 ml potato dextrose broth (PDB) medium was inoculated with lml mycelia suspension of different isolates of Aspergillus ochraceus group and other fungal species as mentioned in table.2. The flasks were incubated at ambient temperature of 26° to 28° C. under stationary conditions for 48-72 h. The mycelia from the culture broth were separated by centrifugation.

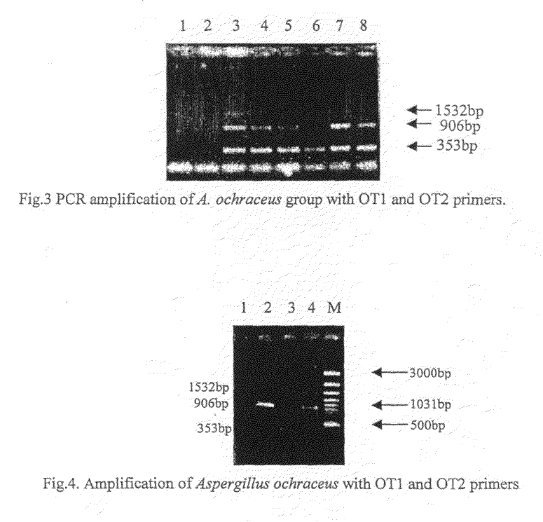

The DNA from the mycelium was extracted for PCR by the method of Lee et al (1998). Amplification was performed in total reaction volume of 25 μl, which contained 1× PCR buffer (10 mM Tris-HCl, pH9.0, 50 mM KCl, 1.5 mM MgCl2, 0.01% gelatin), 12.5 μM each of deoxynucleotide phosphate, 10 pmol of each primer, and one unit of Taq DNA Polymerase, 25 ng template DNA and sterile ultra filtered water. Template DNA was initially denatured at 95° C. for 5 minutes. Subsequently a total of 30 amplification cycles was carried out in a programmable thermocycler with an initial 8 cycles each with a denaturation for 30 sec at 94° C., primer annealing for 45 seconds at 70° C., extension for 75 seconds at 72° C. followed by 22 cycles each with a denaturation for 30 sec at 94° C., primer annealing for 45 seconds at 60° C., extension for 75 seconds at 72° C. and final extension for 8 minutes at 72° C. PCR products were analyzed by standard procedure using agarose gel electrophoresis. Gel was stained with ethidium bromide (0.5 μg/ml), destained with distilled water and examined on a UV-transilluminator. A 100 bp ladder was used as a molecular size marker. The amplification profile of the gel was documented in a CCD-Camera based gel documentation system. The data presented in FIG. 3 indicates specific amplification of the target 18S rDNA gene in Aspergillus ochraceus species by OT1 and OT2 primers yielding 906 bp and 353 bp respectively and amplification product of 1532 bp signifying the interaction of OT1F primer with OT2R primer which shows the specificity of the multiplex PCR. However, none of the other fungal species (data not shown) gave positive amplification in multiplex PCR.

### Example. 4

The example refers to isolation of spores from food sample. Aspergillus ochraceus spores ranging from 110⁶-10⁸ spores/g were inoculated to maize grits in 250 ml flasks. Maize sample (1 g) was suspended in 10 ml distilled water and the spores were separated from the maize grits by low speed centrirfigation (1000×g) for 5 minutes. Supernatant (1 ml) containing spores was transferred to 1.5 ml microcentrifuge tube and centrifuged at 10000×g for 15 minutes and supernatant was discarded. The spores were suspended in 1 ml potato dextrose broth and enriched for 12 h at 30±2° C.

The DNA from the mycelium was extracted for PCR by the method of Lee et al (1998). Amplification was performed in total reaction volume of 25 μl, which contained 1× PCR buffer (10 mM Tris-HCl, pH9.0, 50 mM KCl, 1.5 mM MgCl2, 0.01% gelatin), 12.5 μM each of deoxynucleotide phosphate, 10 pmol of each primer, and one unit of Taq DNA Polymerase, 25 ng template DNA and sterile ultra filtered water. Template DNA was initially denatured at 95° C. for 5 minutes. Subsequently a total of 30 amplification cycles was carried out in a programmable thermocycler with an initial 8 cycles each with a denaturation for 30 sec at 94° C., primer annealing for 45 seconds at 70° C., extension for 75 seconds at 72° C. followed by 22 cycles each with a denaturation for 30 sec at 94° C., primer annealing for 45 seconds at 60° C., extension for 75 seconds at 72° C. and final extension for 8 minutes at 72° C. PCR products were analyzed by standard procedure using agarose gel electrophoresis. Gel was stained with ethidium bromide (0.5 μg/ml), destained with distilled water and examined on a UV-transilluminator. The amplification profile of the gel was documented in a CCD-Camera based gel documentation system. A 100-3000 bp DNA marker was used as a molecular size marker. The data presented in FIG. 4. indicates specific amplification of the target 18S rRNA gene by the primer sets OT1, OT2 and OT1F+OT2R primer in maize inoculated with Aspergillus ochraceus spores.

TABLE 1
List of 18S rDNA sequences of organisms
examined for designing primers.

| Organism | Strain | Source | Accesion Number |
|---|---|---|---|
| Aspergillus ochraceus | NRRL 1642 | DDBJ | AB002068.1 |
| Aspergillus ochraceus | UPSC 1983 | GenBank | AF548065.1 |
| Aspergillus ochraceus | CBS 108.08 | EMBL | AB008405.1 |
| Aspergillus candidus | IAM 13850 | DDBJ | AB002065.1 |
| Aspergillus niger | UPSC 1769 | GenBank | AF548064.1 |
| Aspergillus awamori | IFO4033 | DDBJ | D63695.1 |
| Aspergillus sparsus | IAM 13904 | DDBJ | ABOO2066.1 |
| Aspergillus fumigatus | UPSC 2006 | GenBank | AF548062.1 |
| Aspergillus flavus | UPSC 1768 | GenBank | AF548060.1 |
| Aspergillus tamarii | M143 | DDBJ | AB106338.1 |
| Aspergillus nomius | NRRL 13137 | DDBJ | AB008404.1 |
| Aspergillus sojae | IFO4386 | DDBJ | D63700.1 |
| Aspergillus parasiticus | NFRI1153 | DDBJ | D63699.1 |
| Aspergillus pencillioides | ALI 231 | GenBank | AF548066.1 |
| Aspergillus terreus | CBS 106.25 | EMBL | X78540.1 |
| Aspergillus ustus | NRRL 275 | DDBJ | AB002072.1 |
| Emericilla nidulans | FGSC4 | DDBJ | U77377 |
| Aspergillus nidulans | CBS100.20 | EMBL | X78539.1 |
| Aspergillus silvaticus | ALI 234 | GenBank | AF548067.1 |
| Aspergillus versicolor | UPSC 2027 | GenBank | AF548068.1 |
| Eurotium herbariorum | NRRL 116 | DDBJ | AB002069.1 |
| Eurotium amstelodami | FRR 2792 | DDBJ | AB002076.1 |
| Aspergillus restrictus | FRR 2176 | DDBJ | AB002079.1 |
| Aspergillus Wentii | JCM 2724 | DDBJ | AB002063.1 |
| Aspergillus clavatus | NRRL 1 | DDBJ | AB002070.1 |

TABLE 2
List of cultures.

| Culture | Source |
|---|---|
| Aspergillus ochraceus CFR 221 | Culture collection center, Department of Food |
| | Microbiology, CFTRI, Mysore, India. |
| Aspergillus sclerotiorum CFR227 | Groundnut |
| Aspergillus ostianus CFR228 | Chili |
| Aspergillus melleus CFR 226 | Maize |
| Aspergillus auricomus CFR229 | Dried coconut |
| Aspergillus sulphureus CFR230 | Coffee beans |
| Aspergillus niger CFR 1398 | Culture collection center, Department of Food |
| | Microbiology, CFTRI, Mysore, India.. |
| Aspergillus ochraceus MTCC 1877 | Microbial Type Culture Collection, Institute of |
| | Microbial Technology, Chandigarh, India. |
| Pencillium verrucossum MTCC 2007 | Microbial Type Culture Collection, Institute of |
| | Microbial Technology, Chandigarh, India. |
| Penicillium viridicatum MTCC 1758 | Microbial Type Culture Collection, Institute of |
| | Microbial Technology, Chandigarh, India. |
| Aspergillus oryzae NCIM 665 | NCL, Pune, India |
| Aspergillus oryzae NCIM 645 | ″ |
| Fusarium NCIM 3329 | ″ |
| Aspergillus flavus ATCC 46283 | American Type culture collection, USA |
| Aspergillus parasiticus CFR223 | Culture collection center, Department of Food |
| | Microbiology, CFTRI, Mysore, India. |
| Phoma CFR 231 | Culture collection center, Department of Food |
| | Microbiology, CFTRI, Mysore, India. |
| Rhizopus CFR 234 | Culture collection center, Department of Food |
| | Microbiology, CFTRI, Mysore, India. |
| Saccharomyces cerevisiae CFR 505 | Culture collection center, Department of Food |
| | Microbiology, CFTRI, Mysore, India. |
| Alternaria alternata CFR 233 | Culture collection center, Department of Food |
| | Microbiology, CFTRI, Mysore, India. |
| Aspergillus candidus CFR 234 | Culture collection center, Department of Food |
| | Microbiology, CFTRI, Mysore, India. |

The main advantages of the present invention are
1. The designed set of primers for the 18S rRNA gene is specific for the detection of Aspergillus ochraceus species.
2. The method can be used for rapid, simple and cost effective detection of species belonging to Aspergillus ochraceus group, which are known producers of ochratoxin in pure culture and in food system.

1. A set of novel oligonucleotides primers selected from the group consisting of OT1F, OT1R, OT2F and OT2R having the following respective sequences:
SEQ ID NO: 01
OT1F-5′ GCTAATACATGCTGAAAACCCCA3′
SEQ ID NO: 02
OT1R-5′ GCGGGTCATAATAGAAACACCGC3′
SEQ ID NO: 03
OT2F-5′ TAAATAGCCCGGTCCGCATTCG3′
SEQ ID NO: 04
OT2R-5′ TCCCCTGAGCCAGTCCGAA3′
useful for amplification of target nucleotide fragment forming a part of 18S rRNA gene in species belong to Aspergillus ochraceus (MTCC No: 1877) group.

2. The set of novel oligonucleotide primers according to claim 1, wherein the primer pair OT1F (SEQ ID NO: 01) and OT1R (SEQ ID NO: 02) is useful for amplification of 906 bp nucleotide fragment forming a part of 18S rRNA gene in species belonging to Aspergillus ochraeus (MTCC No: 1877) group.

3. The set of novel oligonucleotide primers according to claim 1, wherein the primer pair OT2F (SEQ ID NO: 03) and OT2R (SEQ ID NO: 02) is useful for amplification of 353 bp nucleotide fragment forming a part of 183 rRNA gene in species belonging to Aspergillus ochraceus (mtcc No: 1877) group.

4. The set of novel oligonucleotide primers as claimed in claims 1, wherein the said amplification is achieved by Polymerase Chain Reaction (PCR), Rolling Circle Amplification (RCA), Strand Displacement Amplification (SDA), Nucleic Acid Sequence Based Amplification (NASBA).

5. The set of novel oligonucleotide primers according to claims 1, wherein the said amplification is useful for various applications selected from the group consisting of detecting, quantifying, screening, quality-monitoring and combination thereof.

6. The set of novel oligonucleotide primers according to claims 1, wherein the said primer sequences and/or parts thereof are useful as probes for the detecting nucleotide fragment forming a part of 18S rRNA gene in species belonging to Aspergillus ochraceus (MTCC No: 1877) group.

7. A method for the detection of Aspergillus ochraceus (MTCC No: 1877) group, using oligonucleotide primers of claims 1, which comprises the steps of:
(a) isolating, by a known method, of DNA from the source sample contaminated with Aspergillus ochraceus (MTCC No: 1877) group.
(b) subjecting the DNA isolated in step (a) to Polymerase Chain Reaction (PCR) amplification using at least one pair of the primers selected from the group consisting of pairs OT2F (SEQ ID NO: 03) and OT2R (SEQ ID NO: 02) OT1F (SEQ ID NO: 01), OT1R (SEQ ID NO: 02) and combination thereof having the following nucleotide sequence:
SEQ ID NO: 01
OT1F-5′ GCTAATACATGCTGAAAACCCCA3′
SEQ ID NO: 02
OT1R-5′ GCGGGTCATAATAGAAACACCGC3′
SEQ ID NO: 03
OT2F-5′ TAAATAGCCCGGTCCGCATTCG3′
SEQ ID NO: 04
OT2R-5′ TCCCCTGAGCCAGTCCGAA3′
useful for amplification of target nucleotide fragment forming a part of 18S rRNA gene in species belonging to Aspergillus ochraceus (MTCC No: 1877) group; and
(c) detecting the Aspergillus ochraceus (MTCC No: 1877) group by visualizing the product(s) of said Polymerase Chain Reaction (PCR) amplification product(s) corresponding to target nucleotide fragment forming a part of 18S rRNA gene in species belong to Aspergillus ochraceus (MTCC No: 1877) group.

8. The method according to claim 7, wherein the said Polymerase Chain Reaction (PCR) amplification used has the following parameters:
PCR reaction mixture: total reaction volume of 25 μl containing 1× PCR buffer (10 mM Tris-HCl, pH 9.0, 50 mM MgCl2, 0.01% gelatin); Deoxyribo nucleoside triphosphate: 12.5 μM each; Primers: 10 pmol each; Template DNA: 25 ng; Taq DNA polymerase: 1 unit; and Sterile ultrafiltered water: added to make up the total reaction volume of 25 μl; and
Thermal parameters: initial denaturation at 90°-98° C. for 2-8 min; initial amplification of 6-10 cycles each with a denaturation at 93°-95° C. for 20-40 sec, annealing at 68° to 72° for 35 to 55 seconds and an extension at 70° to 74° C. for 60 to 90 seconds followed by amplification of 20-24 cycles each with a denaturation at 93°-95° C. for 20-40 sec, annealing at 58° to 62° for 35 to 55 seconds and an extension at 70° to 74° C. for 60 to 90 seconds and a final extension at 68° to 76° C. for 6 to 10 minutes.

9. The method according to claims 7, wherein the said detection is carried out be analysing the PCR products in 1.0-1.2% agarose gel electrophoresis for 1.5-2.5 h at about 100 volts in about 1× TAE buffer in order to get bands; staining the bands with ethidium bromide (o.5 μg/ml) followed by de-staining with distilled water and examination on a UV-transilluminator for appearance of bands, against 100 bp ladder used as molecular weight marker, corresponding to target nucleotide fragment forming a part of 18S rRNA gene in species belonging to Aspergillus ochracaeus (MTCC No: 1877) group.

10. The method according to claims 7, wherein amplification of 18S rRNA gene of Aspergillus ochraceus group is effected by about 30 amplification cycles comprising of about 8 cycles each having denaturation for about 30 sec at about 94° C., primer annealing for about 45 seconds at about 70° C., extension for about 75 seconds at bout 72° C. followed by about 22 cycles each having denaturation for about 30 sec at about 94° C., primer annealing for about 45 seconds at about 60° C., extension for about 75 seconds at about 72° C. and final extension for about 8 minutes at about 72° C.

11. The method according to claim 7, where in the PCR used is performed by multiplex PCR in order to obtain the following amplification products:

| Primers used | Amplification product size |
|---|---|
| OT1F and OT1R | 906 bp |
| OT2F and OT2R | 353 bp |
| OT1F and OT2R | 1532 bp |

12. The method according to claims 7, wherein the source sample used is selected from the group consisting of any food systems, foods, beverages, grains, fruits, vegetables, oil seeds, oil and mixture thereof.

13. The method according to claims 7, wherein the source sample used is selected from the group consisting of any baked- or un-baked foods, food-systems, vegetables, grains, fruits, vegetables, oil seeds, oil and mixture thereof.

14. The method according to claims 7, wherein the source sample used is selected from the group consisting of soil, water, air and mixture thereof.

15. A kit for amplifying or quantifying a nucleic acid target comprising at least one pair of the primers selected from the group consisting of pairs OT2F (SEQ ID NO: 03) and OT2R (SEQ ID NO: 02) OT1F (SEQ ID NO: 01), OT1R (SEQ ID NO: 02) and combination thereof having the following nucleotide sequence:
SEQ ID NO: 01
OT1F-5′ GCTAATACATGCTGAAAACCCCA
SEQ ID NO: 02
OT1R-5′ GCGGGTCATAATAGAAACACCGC
SEQ ID NO: 03
OT2F-5′ TAAATAGCCCGGTCCGCATTCG3′
SEQ ID NO: 04
OT2R-5′ TCCCCTGAGCCAGTCCGAA3′
useful for amplification of target nucleotide fragment forming a part of 18S rRNA gene in species belonging to Aspergillus ochraceus (MTCC No: 1877) group.
